(19) United States
(12) Patent Application Publication
(10) Pub. No.: US 2025/0228761 A1
(43) Pub. Date: Jul. 17, 2025

(54) COMPOSITION COMPRISING A SURFACE-REACTED CALCIUM CARBONATE AND A TANNIN

(71) Applicant: Omya International AG, Oftringen (CH)

(72) Inventors: Julia IVANOV, Binningen (CH); Jamal FTOUNI, Zofingen (CH); Ivan SCHLATTER, Zofingen (CH); Marcel LEX, Hirschau (DE); Florentine Marianne HILTY-VANCURA, Meilen (CH); Joachim SCHOELKOPF, Oberkulm (CH)

(73) Assignee: Omya International AG, Oftringen (CH)

(21) Appl. No.: 18/698,238

(22) PCT Filed: Sep. 29, 2022
(86) PCT No.: PCT/EP2022/077178

§ 371 Date: Apr. 3, 2024

(30) Foreign Application Priority Data
Oct. 4, 2021 (EP) 21200690.2

(51) Int. Cl. A61K 8/60; A61K 8/19; A61Q 15/00

(52) U.S. Cl. CPC A61K 8/602; A61K 8/19; A61Q 15/00; A61K 2800/30

(57) ABSTRACT

The present invention provides a composition comprising (i) a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and (ii) a tannin or a derivative thereof. The composition according to the invention is useful for reducing sweating and/or sweat odor. The present invention further provides a personal care formulation comprising the composition according to the invention.

### TECHNICAL FIELD OF THE INVENTION

The present invention relates to compositions for use in personal care formulations, and particularly deodorant and/or antiperspirant formulations. Furthermore, the present invention relates to compositions for reducing sweating and/or sweat malodor.

### BACKGROUND OF INVENTION

Deodorants and antiperspirants are widely used to regulate sweating and unpleasant odors which may occur when sweat components are converted, e.g. by microorganisms on the skin, into volatile substances. Many commercially available products aim at controlling or suppressing sweating, e.g. axillary sweating, by adding aluminum compounds and/or zirconium compound into the formulation. Aluminum compounds or zirconium compound can block or clog sweat glands or sweat pores when contacted with sweat, and thereby can reduce sweating for a specific period of time as well as unpleasant odor which may come along with sweating.

Over the last years, customers and others have expressed concerns over environmental impact and health risks such as skin irritation in personal care products, and especially in connection with aluminum compounds. In view thereof, there is a continuous need in the art for alternative and/or improved compounds or compositions which can be used to regulate or reduce sweating and/or sweat malodor.

An object of the present invention may be to provide a new and improved composition or personal care formulation which is suitable for reducing sweating and/or sweat malodor.

### SUMMARY OF INVENTION

The present invention provides in one aspect a composition comprising
(i) a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and
(ii) a tannin or a derivative thereof.

In one aspect, the present invention provides a personal care formulation comprising the composition according to the invention.

In one aspect, the present invention provides a use of a composition according to the invention for reducing sweating and/or sweat odor.

The inventors found that a composition comprising a surface-reacted calcium carbonate as defined herein and a tannin or a derivative thereof are useful for reducing sweating and sweat malodor, and are therefore particularly suitable for use in personal care formulations such as deodorants and antiperspirants. The inventive composition may reduce sweating and/or sweat odor more effective compared to e.g. aluminum chlorohydrate which is widely used in the market for such purposes. The inventive composition may be prepared aluminum- and/or zirconium-free, thereby avoiding environmental and/or health concerns which may be associated with such components. Tannins which can be used for preparing the inventive composition are naturally occurring substances, and therefore can be obtained from renewable natural resources.

Furthermore, the inventors unexpectedly found that loading of the tannin or the derivative thereof onto the surface-reacted calcium carbonate leads to a particularly effective antiperspirant and/or deodorant composition.

Further embodiments of the invention are defined in the dependent claims.

According to one embodiment, the composition is a solid composition, and/or the composition essentially consists of at least one surface-reacted calcium carbonate and at least one tannin or derivative thereof.

According to one embodiment, the surface-reacted calcium carbonate is loaded with the tannin or the derivative thereof, optionally by a process comprising the steps of: (a) providing a liquid composition comprising the surface-reacted calcium carbonate and the tannin or the derivative thereof, and (b) drying the liquid composition provided in step (a) to obtain a solid composition.

According to one embodiment, the surface-reacted calcium carbonate and the tannin or the derivative thereof form a solid particle blend.

According to one embodiment, the surface-reacted calcium carbonate and the tannin or the derivative thereof are present in the composition in a weight ratio of from 99:1 to 25:75, and preferably from 95:5 to 35:65.

According to one embodiment, the one or more H₃O⁺ ion donors are selected from the group consisting of hydrochloric acid, sulfuric acid, sulfurous acid, phosphoric acid, citric acid, oxalic acid, an acidic salt, acetic acid, formic acid, and mixtures thereof.

According to one embodiment, the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment.

According to one embodiment, the surface-reacted calcium carbonate has one or more of the following characteristics, and preferably has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm,
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method, and
(iv) an intra-particle intruded specific pore volume in the range from 0.10 to 2.3 cm³/g, more preferably from 0.20 to 2.0 cm³/g, even more preferably from 0.40 to 1.8 cm³/g and most preferably from 0.60 to 1.6 cm³/g, calculated from mercury porosimetry measurement.

According to one embodiment, the surface-reacted calcium carbonate comprises calcium carbonate and hydroxyapatite (HAP), optionally in a weight ratio in the range of 90:10 to 10:90, preferably in the range of 80:20 to 10:90, more preferably in the range of 60:40 to 10:90, even more preferably in the range of 40:60 to 10:90, and most preferably in the range of 25:75 to 10:90.

According to one embodiment, the tannin is selected from the group consisting of gallotannins, mixtures thereof, and derivatives thereof, or the tannin is selected from the group of hydrolysable tannins, mixtures thereof, and derivatives thereof.

According to one embodiment, the tannin is tannic acid or a derivative thereof.

According to one embodiment, the personal care formulation is a deodorant formulation and/or antiperspirant formulation.

According to one embodiment, the personal care formulation is free of aluminum compounds and/or a zirconium compounds.

In the following, the invention is described in more detail.

Where the present description and claims define subject-matter “comprising” certain features, this is to interpreted as meaning that it includes those features, but that it does not exclude other non-specified features. For the purposes of the present invention, the term “essentially consisting of” and “consisting of” are considered to be specific embodiments of the term “comprising of”. If hereinafter a subject-matter is defined to comprise at least a certain number of features, this is also to be understood to disclose a subject-matter, which optionally (essentially) consists only of these features.

Whenever the terms “including” or “having” are used, these terms are meant to be equivalent to “comprising” as defined above.

Where an indefinite or definite article is used when referring to a singular noun, e.g. “a”, “an” or “the”, this includes a plural of that noun unless something else is specifically stated. Terms like “obtainable” or “obtained” are used interchangeably. This e.g. means that, unless the context clearly dictates otherwise, the term “obtained” does not mean to indicate that, an embodiment must be obtained by the sequence of steps following the term “obtained” even though such a limited understanding is always included by the terms “obtained” as a preferred embodiment.

### DETAILED DESCRIPTION OF INVENTION

### Composition According to the Invention

The present invention provides a composition comprising
(i) a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and
(ii) a tannin or a derivative thereof.

### The Surface-Reacted Calcium Carbonate

The composition according to the invention comprises a component (i) which is a surface-reacted calcium carbonate. The surface-reacted calcium carbonate is a reaction product of ground natural calcium carbonate (GNCC) or precipitated calcium carbonate (PCC) with carbon dioxide and one or more H₃O⁺ ion donors. The carbon dioxide is formed in situ by the H₃O⁺ ion donors treatment and/or is supplied from an external source.

A “H₃O⁺ ion donor” in the context of the present invention is a Brønsted acid and/or an acid salt.

In a preferred embodiment of the invention the surface-reacted calcium carbonate is obtained by a process comprising the steps of: (a) providing a suspension of natural or precipitated calcium carbonate, (b) adding at least one acid having a pKₐ value of 0 or less at 20° C. or having a pKₐ value from 0 to 2.5 at 20° C. to the suspension of step (a), and (c) treating the suspension of step (a) with carbon dioxide before, during or after step (b). According to another embodiment the surface-reacted calcium carbonate is obtained by a process comprising the steps of: (A) providing a natural or precipitated calcium carbonate, (B) providing at least one water-soluble acid, (C) providing gaseous CO₂, (D) contacting said natural or precipitated calcium carbonate of step (A) with the at least one acid of step (B) and with the CO₂ of step (C), characterised in that: (i) the at least one acid of step B) has a pKₐ of greater than 2.5 and less than or equal to 7 at 20° C., associated with the ionisation of its first available hydrogen, and a corresponding anion is formed on loss of this first available hydrogen capable of forming a water-soluble calcium salt, and (ii) following contacting the at least one acid with natural or precipitated calcium carbonate, at least one water-soluble salt, which in the case of a hydrogen-containing salt has a pKₐ of greater than 7 at 20° C., associated with the ionisation of the first available hydrogen, and the salt anion of which is capable of forming water-insoluble calcium salts, is additionally provided.

“Ground natural calcium carbonate” is abbreviated herein as GNCC or GCC. The abbreviations are used interchangeably. Ground natural calcium carbonate (GNCC) preferably is selected from calcium carbonate containing minerals selected from the group comprising marble, chalk, limestone and mixtures thereof. Natural calcium carbonate may comprise further naturally occurring components such as alumino silicate etc.

In general, the grinding of ground natural calcium carbonate may be a dry or wet grinding step and may be carried out with any conventional grinding device, for example, under conditions such that comminution predominantly results from impacts with a secondary body, i.e. in one or more of: a ball mill, a rod mill, a vibrating mill, a roll crusher, a centrifugal impact mill, a vertical bead mill, an attrition mill, a pin mill, a hammer mill, a pulveriser, a shredder, a de-clumper, a knife cutter, or other such equipment known to the skilled man. In case the calcium carbonate containing mineral material comprises a wet ground calcium carbonate containing mineral material, the grinding step may be performed under conditions such that autogenous grinding takes place and/or by horizontal ball milling, and/or other such processes known to the skilled man. The wet processed ground calcium carbonate containing mineral material thus obtained may be washed and dewatered by well-known processes, e.g. by flocculation, filtration or forced evaporation prior to drying. The subsequent step of drying (if necessary) may be carried out in a single step such as spray drying, or in at least two steps. It is also common that such a mineral material undergoes a beneficiation step (such as a flotation, bleaching or magnetic separation step) to remove impurities.

“Precipitated calcium carbonate” (PCC) in the meaning of the present invention is a synthesized material, generally obtained by precipitation following reaction of carbon dioxide and calcium hydroxide in an aqueous environment or by precipitation of calcium and carbonate ions, for example CaCl₂) and Na₂CO₃, out of solution. Further possible ways of producing PCC are the lime soda process, or the Solvay process in which PCC is a by-product of ammonia production. Precipitated calcium carbonate exists in three primary crystalline forms: calcite, aragonite and vaterite, and there are many different polymorphs (crystal habits) for each of these crystalline forms. Calcite has a trigonal structure with typical crystal habits such as scalenohedral (S-PCC), rhombohedral (R-PCC), hexagonal prismatic, pinacoidal, colloidal (C-PCC), cubic, and prismatic (P-PCC). Aragonite is an orthorhombic structure with typical crystal habits of twinned hexagonal prismatic crystals, as well as a diverse assortment of thin elongated prismatic, curved bladed, steep pyramidal, chisel shaped crystals, branching tree, and coral or worm-like form. Vaterite belongs to the hexagonal crystal system. The obtained PCC slurry can be mechanically dewatered and dried.

According to one embodiment of the present invention, the precipitated calcium carbonate is precipitated calcium carbonate, preferably comprising aragonitic, vateritic or calcitic mineralogical crystal forms or mixtures thereof.

Precipitated calcium carbonate may be ground prior to the treatment with carbon dioxide and at least one H₃O⁺ ion donor by the same means as used for grinding natural calcium carbonate as described above.

According to one embodiment of the present invention, the natural or precipitated calcium carbonate is in form of particles having a weight median particle size dso of 0.05 to 10.0 μm, preferably 0.2 to 5.0 μm, more preferably 0.4 to 3.0 μm, most preferably 0.6 to 1.2 μm, especially 0.7 μm. According to a further embodiment of the present invention, the natural or precipitated calcium carbonate is in form of particles having a top cut particle size d₉₈ of 0.15 to 55 μm, preferably 1 to 40 μm, more preferably 2 to 25 μm, most preferably 3 to 15 μm, especially 4 μm.

The natural and/or precipitated calcium carbonate may be used dry or suspended in water. Preferably, a corresponding slurry has a content of natural or precipitated calcium carbonate within the range of 1 wt.-% to 90 wt.-%, more preferably 3 wt.-% to 60 wt.-%, even more preferably 5 wt.-% to 40 wt.-%, and most preferably 10 wt.-% to 25 wt.-% based on the weight of the slurry.

The one or more H₃O⁺ ion donor used for the preparation of surface reacted calcium carbonate may be any strong acid, medium-strong acid, or weak acid, or mixtures thereof, generating H₃O⁺ ions under the preparation conditions. According to the present invention, the at least one H₃O⁺ ion donor can also be an acidic salt, generating H₃O⁺ ions under the preparation conditions.

According to one embodiment, the at least one H₃O⁺ ion donor is a strong acid having a pKₐ of 0 or less at 20° C.

According to another embodiment, the at least one H₃O⁺ ion donor is a medium-strong acid having a pKₐ value from 0 to 2.5 at 20° C. If the pKₐ at 20° C. is 0 or less, the acid is preferably selected from sulfuric acid, hydrochloric acid, or mixtures thereof. If the pKₐ at 20° C. is from 0 to 2.5, the H₃O⁺ ion donor is preferably selected from H₂SO₃, H₃PO₄, oxalic acid, or mixtures thereof. The at least one H₃O⁺ ion donor can also be an acidic salt, for example, HSO₄ or H₂PO₄, being at least partially neutralized by a corresponding cation such as Li⁺, Na⁺ or K⁺, or HPO₄²⁻, being at least partially neutralised by a corresponding cation such as Li⁺, Na⁺, K⁺, Mg²⁺ or Ca²⁺. The at least one H₃O⁺ ion donor can also be a mixture of one or more acids and one or more acidic salts.

According to still another embodiment, the at least one H₃O⁺ ion donor is a weak acid having a pKₐ value of greater than 2.5 and less than or equal to 7, when measured at 20° C., associated with the ionisation of the first available hydrogen, and having a corresponding anion, which is capable of forming water-soluble calcium salts. Subsequently, at least one water-soluble salt, which in the case of a hydrogen-containing salt has a pKₐ of greater than 7, when measured at 20° C., associated with the ionisation of the first available hydrogen, and the salt anion of which is capable of forming water-insoluble calcium salts, is additionally provided. According to the preferred embodiment, the weak acid has a pKₐ value from greater than 2.5 to 5 at 20° C., and more preferably the weak acid is selected from the group consisting of acetic acid, formic acid, propanoic acid, and mixtures thereof. Exemplary cations of said water-soluble salt are selected from the group consisting of potassium, sodium, lithium and mixtures thereof. In a more preferred embodiment, said cation is sodium or potassium. Exemplary anions of said water-soluble salt are selected from the group consisting of phosphate, dihydrogen phosphate, monohydrogen phosphate, oxalate, silicate, mixtures thereof and hydrates thereof. In a more preferred embodiment, said anion is selected from the group consisting of phosphate, dihydrogen phosphate, monohydrogen phosphate, mixtures thereof and hydrates thereof. In a most preferred embodiment, said anion is selected from the group consisting of dihydrogen phosphate, monohydrogen phosphate, mixtures thereof and hydrates thereof. Water-soluble salt addition may be performed dropwise or in one step. In the case of drop wise addition, this addition preferably takes place within a time period of 10 minutes. It is more preferred to add said salt in one step.

According to one embodiment of the present invention, the at least one H₃O⁺ ion donor is selected from the group consisting of hydrochloric acid, sulfuric acid, sulfurous acid, phosphoric acid, citric acid, oxalic acid, acetic acid, formic acid, and mixtures thereof. Preferably the at least one H₃O⁺ ion donor is selected from the group consisting of hydrochloric acid, sulfuric acid, sulfurous acid, phosphoric acid, oxalic acid, H₂PO₄, being at least partially neutralised by a corresponding cation such as Li⁺, Na⁺ or K⁺, HPO₄²⁻, being at least partially neutralised by a corresponding cation such as Li⁺, Na⁺, K⁺, Mg²⁺, or Ca²⁺ and mixtures thereof, more preferably the at least one acid is selected from the group consisting of hydrochloric acid, sulfuric acid, sulfurous acid, phosphoric acid, oxalic acid, or mixtures thereof, and most preferably, the at least one H₃O⁺ ion donor is phosphoric acid.

The one or more H₃O⁺ ion donor can be added to the suspension as a concentrated solution or a more diluted solution. Preferably, the molar ratio of the H₃O⁺ ion donor to the natural or precipitated calcium carbonate is from 0.01 to 4, more preferably from 0.02 to 2, even more preferably 0.05 to 1 and most preferably 0.1 to 0.58.

As an alternative, it is also possible to add the H₃O⁺ ion donor to the water before the natural or precipitated calcium carbonate is suspended.

In a next step, the natural or precipitated calcium carbonate is treated with carbon dioxide. If a strong acid such as sulfuric acid or hydrochloric acid is used for the H₃O⁺ ion donor treatment of the natural or precipitated calcium carbonate, the carbon dioxide is automatically formed. Alternatively or additionally, the carbon dioxide can be supplied from an external source.

H₃O⁺ ion donor treatment and treatment with carbon dioxide can be carried out simultaneously which is the case when a strong or medium-strong acid is used. It is also possible to carry out H₃O⁺ ion donor treatment first, e.g. with a medium strong acid having a pKₐ in the range of 0 to 2.5 at 20° C., wherein carbon dioxide is formed in situ, and thus, the carbon dioxide treatment will automatically be carried out simultaneously with the H₃O⁺ ion donor treatment, followed by the additional treatment with carbon dioxide supplied from an external source.

In a preferred embodiment, the H₃O⁺ ion donor treatment step and/or the carbon dioxide treatment step are repeated at least once, more preferably several times. According to one embodiment, the at least one H₃O⁺ ion donor is added over a time period of at least about 5 min, preferably at least about 10 min, typically from about 10 to about 20 min, more preferably about 30 min, even more preferably about 45 min, and sometimes about 1 h or more.

Subsequent to the H₃O⁺ ion donor treatment and carbon dioxide treatment, the pH of the aqueous suspension, measured at 20° C., naturally reaches a value of greater than 6.0, preferably greater than 6.5, more preferably greater than 7.0, even more preferably greater than 7.5, thereby preparing the surface-reacted natural or precipitated calcium carbonate as an aqueous suspension having a pH of greater than 6.0, preferably greater than 6.5, more preferably greater than 7.0, even more preferably greater than 7.5.

In a particular preferred embodiment the surface reacted calcium carbonate is a reaction product of ground natural calcium carbonate (GNCC) with carbon dioxide and phosphoric acid, wherein the carbon dioxide is formed in situ by the phosphoric acid treatment.

Further details about the preparation of the surface-reacted natural calcium carbonate are disclosed in WO 00/39222 A1, WO 2004/083316 A1, WO 2005/121257 A2, WO 2009/074492 A1, EP 2 264 108 A1, EP 2 264 109 A1 and US 2004/0020410 A1, the content of these references herewith being included in the present application.

Similarly, surface-reacted precipitated calcium carbonate is obtained. As can be taken in detail from WO 2009/074492 A1, surface-reacted precipitated calcium carbonate is obtained by contacting precipitated calcium carbonate with H₃O⁺ ions and with anions being solubilized in an aqueous medium and being capable of forming water-insoluble calcium salts, in an aqueous medium to form a slurry of surface-reacted precipitated calcium carbonate, wherein said surface-reacted precipitated calcium carbonate comprises an insoluble, at least partially crystalline calcium salt of said anion formed on the surface of at least part of the precipitated calcium carbonate.

Said solubilized calcium ions correspond to an excess of solubilized calcium ions relative to the solubilized calcium ions naturally generated on dissolution of precipitated calcium carbonate by H₃O⁺ ions, where said H₃O⁺ ions are provided solely in the form of a counterion to the anion, i.e. via the addition of the anion in the form of an acid or non-calcium acid salt, and in absence of any further calcium ion or calcium ion generating source.

Said excess solubilized calcium ions are preferably provided by the addition of a soluble neutral or acid calcium salt, or by the addition of an acid or a neutral or acid non-calcium salt which generates a soluble neutral or acid calcium salt in situ.

Said H₃O⁺ ions may be provided by the addition of an acid or an acid salt of said anion, or the addition of an acid or an acid salt which simultaneously serves to provide all or part of said excess solubilized calcium ions.

The surface-reacted calcium carbonate can be kept in suspension, optionally further stabilised by a dispersant. Conventional dispersants known to the skilled person can be used. A preferred dispersant is comprised of polyacrylic acids and/or carboxymethylcelluloses.

Alternatively, the aqueous suspension described above can be dried, thereby obtaining the solid (i.e. dry or containing as little water that it is not in a fluid form) surface-reacted natural or precipitated calcium carbonate in the form of granules or a powder.

In a preferred embodiment, the surface-reacted calcium carbonate has a specific surface area of from 15 m²/g to 200 m²/g, preferably from 27 m2/g to 180 m2/g, more preferably from 30 m2/g to 160 m2/g, even more preferably from 45 m2/g to 150 m2/g, most preferably from 48 m2/g to 140 m²/g, measured using nitrogen and the BET method. For example, the surface-reacted calcium carbonate can have a specific surface area of from 75 m2/g to 120 m2/g (e.g. 85 to 110 m²/g or 90 to 100 m²/g), measured using nitrogen and the BET method. The BET specific surface area in the meaning of the present invention is defined as the surface area of the particles divided by the mass of the particles. As used therein the specific surface area is measured by adsorption using the BET isotherm (ISO 9277:2010) and is specified in m2/g.

It is furthermore preferred that the surface-reacted calcium carbonate particles have a volume median particle size d₅₀ (vol) of from 1 to 75 μm, preferably from 2 to 50 μm, more preferably 3 to 40 μm, even more preferably from 4 to 30 μm, and most preferably from 5 to 15 μm. For example, the surface-reacted calcium carbonate particles may have a volume median particle size d₅₀ (vol) of from 10 to 12 μm (e.g. from 6 to 10 μm).

It may furthermore be preferred that the surface-reacted calcium carbonate particles have a top cut volume particle size d₉₈ (vol) of from 2 to 150 μm, preferably from 4 to 100 μm, more preferably 6 to 80 μm, even more preferably from 8 to 60 μm, and most preferably from 10 to 30 μm. For example, the surface-reacted calcium carbonate particles may have a top cut volume particle size d₉₈ (vol) of from 15 to 25 μm (e.g. from 18 to 22 μm).

It is furthermore preferred that the surface-reacted calcium carbonate particles have a volume median particle size d₅₀ (vol) of from 1 to 75 μm, preferably from 2 to 50 μm, more preferably 3 to 40 μm, even more preferably from 4 to 30 μm, and most preferably from 5 to 15 μm. For example, the surface-reacted calcium carbonate particles may have a volume median particle size d₅₀ (vol) of from 10 to 12 μm (e.g. from 6 to 10 μm).

It may furthermore be preferred that the surface-reacted calcium carbonate particles have a top cut volume particle size d₉₈ (vol) of from 2 to 150 μm, preferably from 4 to 100 μm, more preferably 6 to 80 μm, even more preferably from 8 to 60 μm, and most preferably from 10 to 30 μm. For example, the surface-reacted calcium carbonate particles may have a top cut volume particle size d₉₈ (vol) of from 15 to 25 μm (e.g. from 18 to 22 μm).

The value dₓ represents the diameter relative to which x % of the particles have diameters less than dₓ. This means that the d₉₈ value is the particle size at which 98% of all particles are smaller. The d₉₈ value is also designated as “top cut”. The dₓ values may be given in volume or weight percent. The d₅₀ (wt) value is thus the weight median particle size, i.e. 50 wt.-% of all grains are smaller than this particle size, and the d₅₀ (vol) value is the volume median particle size, i.e. 50 vol.-% of all grains are smaller than this particle size.

Volume median particle size d₅₀ or top cut volume particle size d₉₈ can be evaluated using laser diffraction, e.g. a Malvern Mastersizer 2000 or 3000 Laser Diffraction System. The d₅₀ or d₉₈ value, measured using a Malvern Mastersizer 2000 or 3000 Laser Diffraction System, indicates a diameter value such that 50% or 98% by volume, respectively, of the particles have a diameter of less than this value. The raw data obtained by the measurement can be analysed using the Mie theory, with a particle refractive index of 1.57 and an absorption index of 0.005.

The weight median particle size d₅₀ can be determined by the sedimentation method, which is an analysis of sedimentation behaviour in a gravimetric field. The measurement may be made with a Sedigraph™ 5100 or 5120, Micromeritics Instrument Corporation. The method and the instrument are known to the skilled person and are commonly used to determine grain size of fillers and pigments. The measurement may be carried out in an aqueous solution of 0.1 wt.-% Na₄P₂O₇. The samples can be dispersed using a high-speed stirrer and sonicated.

Preferably, the surface-reacted calcium carbonate has an intra-particle intruded specific pore volume in the range from 0.1 to 2.3 cm³/g, more preferably from 0.2 to 2.0 cm³/g, especially preferably from 0.4 to 1.8 cm³/g and most preferably from 0.6 to 1.6 cm³/g, calculated from mercury porosimetry measurement. For example, the surface-reacted calcium carbonate can have an intra-particle intruded specific pore volume in the range from 0.8 to 1.4 cm³/g (e.g. from 1.0 to 1.25 cm³/g or from 1.1 to 1.25 cm³/g), calculated from mercury porosimetry measurement.

The intra-particle pore size of the surface-reacted calcium carbonate preferably is in a range of from 0.001 to 1.6 μm, more preferably in a range of from 0.001 to 1.3 μm, especially preferably from 0.001 to 1.15 μm and most preferably of 0.001 to 1.0 μm (e.g. 0.002 to 0.60 μm), determined by mercury porosimetry measurement.

The specific pore volume of the surface-reacted calcium carbonate can be measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm (˜nm). The equilibration time used at each pressure step may be 20 seconds. The sample material can be sealed in a 5 cm³ chamber powder penetrometer for analysis. The data can be corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine interparticle packing of the particles themselves. If they also have intraparticle pores, then this region appears bi modal, and by taking the specific pore volume intruded by mercury into pores finer than the modal turning point, i.e. finer than the bi-modal point of inflection, the specific intraparticle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, can be revealed. The differential curves clearly show the coarse agglomerate pore structure region, the interparticle pore region and the intraparticle pore region, if present. Knowing the intraparticle pore diameter range it is possible to subtract the remainder interparticle and interagglomerate pore volume from the total pore volume to deliver the desired pore volume of the internal pores alone in terms of the pore volume per unit mass (specific pore volume). The same principle of subtraction, of course, applies for isolating any of the other pore size regions of interest.

According to one embodiment, the surface-reacted calcium carbonate has one or more of the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm,
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method, and
(iv) an intra-particle intruded specific pore volume in the range from 0.10 to 2.3 cm³/g, more preferably from 0.20 to 2.0 cm³/g, even more preferably from 0.40 to 1.8 cm³/g and most preferably from 0.60 to 1.6 cm³/g, calculated from mercury porosimetry measurement.

According to one embodiment, the surface-reacted calcium carbonate has a specific surface area (BET) from 10 to 200 m²/g, as measured by the BET method.

According to one embodiment, the surface-reacted calcium carbonate has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm, and
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm.

According to one embodiment, the surface-reacted calcium carbonate has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm, and
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method.

According to one embodiment, the surface-reacted calcium carbonate has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm,
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method, and
(iv) an intra-particle intruded specific pore volume in the range from 0.10 to 2.3 cm³/g, more preferably from 0.20 to 2.0 cm³/g, even more preferably from 0.40 to 1.8 cm³/g and most preferably from 0.60 to 1.6 cm³/g, calculated from mercury porosimetry measurement.

According to one embodiment, the surface-reacted calcium carbonate has the following characteristics:
(i) a volume median particle size (d₅₀) from 5 to 15 μm,
(ii) a top cut volume particle size (d₉₈) from 10 to 30 μm,
(iii) a specific surface area (BET) from 48 to 140 m²/g, as measured by the BET method, and
(iv) an intra-particle intruded specific pore volume in the range from 0.60 to 1.6 cm³/g, calculated from mercury porosimetry measurement.

According to one embodiment, the surface-reacted calcium carbonate has the following characteristics:
(i) a volume median particle size (d₅₀) from 6 to 12 μm,
(ii) a top cut volume particle size (d₉₈) from 15 to 25 μm,
(iii) a specific surface area (BET) from 80 to 120 m2/g, as measured by the BET method, and
(iv) an intra-particle intruded specific pore volume in the range from 1.0 to 1.4 cm³/g, calculated from mercury porosimetry measurement.

According to one embodiment, the surface-reacted calcium carbonate comprises hydroxyapatite (HAP).

According to one embodiment, the surface-reacted calcium carbonate comprises calcium carbonate and hydroxyapatite (HAP) in a weight ratio in the range of 90:10 to 10:90, preferably in the range of 80:20 to 10:90, more preferably in the range of 60:40 to 10:90, even more preferably in the range of 40:60 to 10:90, and most preferably in the range of 25:75 to 10:90.

According to one embodiment, the surface-reacted calcium carbonate comprises (i) calcium carbonate in an amount in the range of 5 to 80 wt. %, preferably in an amount of 10 to 60 wt. %, more preferably in the range of 10 to 40 wt. %, and most preferably in an amount of 10 to 25 wt. %, and (ii) hydroxyapatite (HAP) in an amount in the range of 20 to 90 wt. %, preferably 40 to 90 wt. %, more preferably 60 to 90 wt. %, and most preferably in the range of 75 to 90 wt. %, based on the total weight of the surface-reacted calcium carbonate.

According to one embodiment, the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment, wherein the surface-reacted calcium carbonate has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm,
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method, and
(iv) an intra-particle intruded specific pore volume in the range from 0.10 to 2.3 cm³/g, more preferably from 0.20 to 2.0 cm³/g, even more preferably from 0.40 to 1.8 cm³/g and most preferably from 0.60 to 1.6 cm³/g, calculated from mercury porosimetry measurement,
and wherein the surface-reacted calcium carbonate comprises hydroxyapatite (HAP) in a weight ratio in the range of 80:20 to 10:90, preferably in the range of 60:40 to 10:90, more preferably in the range of 40:60 to 10:90, and most preferably in the range of 25:75 to 10:90.

According to one embodiment, the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment, wherein the surface-reacted calcium carbonate has the following characteristics:
(i) a volume median particle size (d₅₀) from 6 to 12 μm,
(ii) a top cut volume particle size (d₉₈) from 15 to 25 μm,
(iii) a specific surface area (BET) from 80 to 120 m2/g, as measured by the BET method, and
(iv) an intra-particle intruded specific pore volume in the range from 1.0 to 1.4 cm³/g, calculated from mercury porosimetry measurement,
and wherein the surface-reacted calcium carbonate comprises hydroxyapatite (HAP) in the range of 25:75 to 10:90.

The surface-reacted calcium carbonate preferably does not contain an aluminum content of more than 1000 ppm, preferably more than 300 ppm, and more preferably of more than 200 ppm.

### The Tannin or the Derivative Thereof

The composition according to the invention comprises a component (ii) which is a tannin or a derivative of a tannin. Preferably, component (ii) is a tannin.

The term “tannin” refers to a group of naturally occurring substances, and should not be construed as referring only to the specific substance of tannic acid which is however part of the group of tannins. Although tannins occur naturally, it is also possible to synthesize tannins and the term “tannin” is to be understood as covering tannins obtained from natural sources (e.g. by extraction) and obtained by synthetic methodology.

Tannins are well-known to the person of skill in the art. A tannin can be defined as a water-soluble polyphenol having a molecular weight in the range of 500 to 20 000 g/mol. “Water-soluble” in this context can mean a water solubility at 25° C. of more than 10 g (substance)/L (water), and preferably 100 g (substance)/L (water).

Tannins can be classified in four different subgroups: complex tannins, condensed tannins, gallotannins, and ellagitannins (see: K. Khnababaee and T. van Ree, “Tannins: Classification and Definition”, Nat. Prod. Rep., 2001, 18, 641-649). The different subgroups of tannins are known to the person of skill.

“Complex tannins” can be defined as a tannin comprising a catechin unit which is bound glycosidically to a gallotannin or a ellagitannin unit.

“Condensed tannins” can be defined as oligomeric or polymeric proanthocyanidins formed by linkage of C-4 of one catechin with C-8 or C-6 of another monomeric catechin unit.

“Ellagitannins” can be defined as a tannin in which at least two galloyl units are C—C coupled to each other, and which does not contain a glycosidically linked catechin unit.

“Gallotannins” can be defined as a tannin comprising one or more galloyl units and/or one or more of their meta-depsidic derivatives (e.g. meta-di- or meta-trigalloyl units), which are bound to a polyol-, catechin or triterpenoid unit. In this context, “meta-depsidic derivatives of the galloyl unit” is a galloyl unit which is bound (at the meta-position with respect to the carboxyl group of the galloyl unit) by an ester to a galloyl unit, a digalloyl unit, or a trigalloyl unit, etc.

Selected gallotannins and ellagitannins can be grouped together as “hydrolysable tannins” due to their similar chemical reactivity towards hydrolysis. “Hydrolysable tannins” can be defined as gallotannins and ellagitannins which can be hydrolyzed to form gallic acid and/or ellagic acid. According to one embodiment, the tannin is selected from the group consisting of gallotannins, ellagitannins, mixtures thereof, and derivatives thereof. According to one embodiment, the tannin is selected from the group consisting of hydrolysable tannins, mixtures thereof, and derivatives thereof.

According to one embodiment, the tannin is selected from the group consisting of gallotannins, mixtures thereof, and derivatives thereof. The tannin may be a gallotannin or derivative thereof, wherein the gallotannin comprises one or more galloyl units and/or one or more of their meta-depsidic derivatives (e.g. meta-di- or meta-trigalloyl units), which are bound to a polyol unit (e.g. a carbohydrate).

Preferably, the tannin may be a gallotannin or derivative thereof, wherein the gallotannin comprises one or more galloyl units and/or one or more meta-di- or meta-trigalloyl units, which are bound to a polyol unit, and preferably a carbohydrate (e.g. glucose). The one or more galloyl units and/or one or more meta-di- or meta-trigalloyl units are preferably bound to the polyol unit by one or more ester groups.

Preferably, the tannin may be a gallotannin or derivative thereof, wherein the gallotannin consists of one or more galloyl units and/or one or more meta-di- or meta-trigalloyl units, which are bound to a polyol unit, and preferably a carbohydrate (e.g. a monosaccharide such as glucose), by one or more ester groups.

According to one embodiment, the tannin is a gallotannin or derivative thereof, wherein the gallotannin consists of one or more galloyl units and/or one or more meta-digalloyl units, which are bound to glucose, by one or more ester groups.

According to one embodiment, the tannin is a gallotannin or derivative thereof, wherein the gallotannin consists of one or more galloyl units and/or one or more meta-digalloyl units, which are bound to glucose, by one or more ester groups.

According to one embodiment, the tannin is a gallotannin or derivative thereof, wherein the gallotannin consists of one or more meta-digalloyl units, which are bound to glucose, by one or more ester groups.

In each one of the above embodiment, the combined one or more units bound to the polyol (e.g. carbohydrate, monosaccharide or glucose) may be one to five units.

According to one preferred embodiment, the tannin is tannic acid or a derivative thereof.

Tannic acid has the CAS number 1401-55-4. Tannic acid has the following formula:

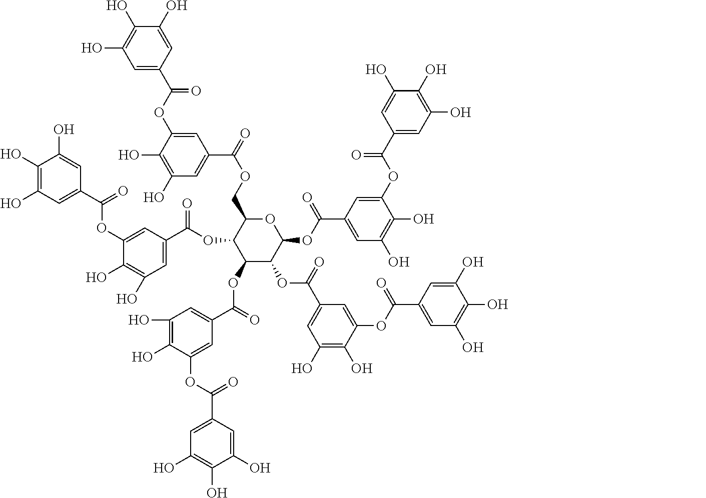 [SMILES: O=C(OC[C@H]1O[C@@H](OC(=O)c2cc(O)c(O)c(OC(=O)c3cc(O)c(O)c(O)c3)c2)[C@H](OC(=O)c2cc(O)c(O)c(OC(=O)c3cc(O)c(O)c(O)c3)c2)[C@@H](OC(=O)c2cc(O)c(O)c(OC(=O)c3cc(O)c(O)c(O)c3)c2)[C@@H]1OC(=O)c1cc(O)c(O)c(OC(=O)c2cc(O)c(O)c(O)c2)c1)c1cc(O)c(O)c(OC(=O)c2cc(O)c(O)c(O)c2)c1]

The tannic acid may be derived from a natural source such as chestnut extract.

According to one embodiment, the tannin or the derivative thereof forms a precipitate when contacted with sweat or an artificial sweat composition. For example, the tannin or its derivative can form a precipitate when tested in the following protocol:
contacting the tannin or its derivative with artificial sweat composition (e.g. NaCl 0.5%, lactic acid 0.1%, urea 0.1%, bovine serum albumin 0.1%, remainder H₂O; pH=6.5) at 20-25° C. using an 0.1-5% (w/v) solution of tannin or its derivative,
observing the formation of a precipitate (e.g. by visual inspection).

The tannin or its derivative can have the form of a particulate matter such as a powder.

### The Inventive Composition

The composition according to the invention may be a solid composition or a liquid composition.

According to one embodiment, the composition is a liquid composition. According to one embodiment, the composition is an aqueous suspension or an aqueous dispersion.

According to one preferred embodiment, the composition is a solid composition.

The solid composition may be a solid particulate blend comprising the surface-reacted calcium carbonate and the tannin or its derivative. The surface-reacted calcium carbonate and the tannin or derivative thereof can form a solid particle blend. The solid particle blend can be obtained by a process comprising the steps of: (i) providing the surface-reacted calcium carbonate in solid form and the tannin or its derivative in solid form, and (ii) mixing the solids to obtain a solid particle blend (e.g. a powder blend).

It is preferred that the surface-reacted calcium carbonate is loaded with the tannin or the derivative thereof. In this context, “loaded” means that the tannin or its derivative is absorbed and/or adsorbed onto the surface of the surface-reacted calcium carbonate.

Different processes are available for loading the surface-reacted calcium carbonate with the tannin or the derivative thereof.

The surface-reacted calcium carbonate can be loaded with the tannin or its derivative by a process comprising the steps of: (a) providing a liquid composition comprising the surface-reacted calcium carbonate and the tannin or the derivative thereof, and (b) drying the liquid composition provided in step (a) to obtain a solid composition. The liquid composition provided in step (a) may comprise water and/or an organic solvent such as an alcohol (e.g. ethanol). Preferably, the liquid composition provided in step (a) is an aqueous compositions, optionally comprising an organic co-solvent such as an alcohol (e.g. ethanol). For example, the liquid composition may be a mixture of water and ethanol.

The solvent of the liquid composition provided in step (a) can be selected to achieve a high solubility of the tannin or its derivative. Preferably, the liquid composition provided in step (a) comprises the tannin or its derivative in essentially fully dissolved form (e.g, wherein the tannin or its derivative are dissolved by at least 95%).

The liquid composition provided in step (a) may be prepared, e.g. by a mixing step, at ambient temperature (e.g. 15 to 25° C.) or at elevated temperatures (e.g. above 30° C. or above 50° C.). Drying step (b) can be carried out by any drying method which is suitable for drying a liquid composition, and preferably for drying an aqueous suspension comprising a mineral material. Preferably, step (b) is carried out by spray drying.

Preferably, the surface-reacted calcium carbonate can be loaded with the tannin or its derivative by a process comprising the steps of: (a) providing an aqueous composition comprising the surface-reacted calcium carbonate and the tannin or the derivative thereof, and (b) spray drying the aqueous composition provided in step (a) to obtain a solid composition.

Additionally or alternatively, the surface-reacted calcium carbonate can be loaded with the tannin or the derivative thereof by incipient wetness impregnation.

According to one embodiment, the composition is a solid composition, wherein the surface-reacted calcium carbonate is loaded with the tannin or the derivative thereof.

In case the surface-reacted calcium carbonate is loaded with the tannin or derivative thereof, the tannin-loaded surface-reacted calcium carbonate can have the form of solid particulate matter. According to one embodiment, the surface-reacted calcium carbonate and the tannin or the derivative thereof together form a solid composite particle. A “composite particle” means that components (i) and (ii) of the inventive composition are together present in a solid particle.

The tannin-loaded surface-reacted calcium carbonate or the solid composite particle formed from tannin and surface-reacted calcium carbonate can have specific physical properties such as a specific particle size and/or specific surface area.

According to one embodiment, the tannin-loaded surface-reacted calcium carbonate has a volume median particle size d₅₀ in the range of from 1.0 to 75 μm, preferably from 2 to 50 μm, more preferably 3 to 40 μm, even more preferably from 4 to 30 μm, and most preferably from 5 to 15 μm, and/or a top cut volume particle size (d₉₈) from 2 to 150 μm, preferably from 4 to 100 μm, more preferably 6 to 80 μm, even more preferably from 8 to 60 μm, and most preferably from 10 to 30 μm.

According to one embodiment, the tannin-loaded surface-reacted calcium carbonate has a specific surface area (BET) from 5 to 150 m2/g, preferably of from 8 to 90 m2/g, more preferably from 10 to 80 m2/g, even more preferably 15 to 75 m2/g, and most preferably from 25 to 75 m2/g, as measured by the BET method.

According to one embodiment, the solid composite particle has a volume median particle size d₅₀ in the range of from 1.0 to 75 μm, preferably from 2 to 50 μm, more preferably 3 to 40 μm, even more preferably from 4 to 30 μm, and most preferably from 5 to 15 μm, and/or a top cut volume particle size (d₉₈) from 2 to 150 μm, preferably from 4 to 100 μm, more preferably 6 to 80 μm, even more preferably from 8 to 60 μm, and most preferably from 10 to 30 μm.

According to one embodiment, the solid composite particle has a specific surface area (BET) from 5 to 150 m2/g, preferably of from 8 to 90 m2/g, more preferably from 10 to 80 m2/g, even more preferably 15 to 75 m2/g, and most preferably from 25 to 75 m2/g, as measured by the BET method.

The composition according to the invention can comprise one surface-reacted calcium carbonate or a mixture of two or more surface-reacted calcium carbonates. Likewise, the composition according to the invention can comprise one tannin or derivative thereof or a mixture of two or more tannins or derivatives of tannins.

The composition according to the invention can comprise the surface-reacted calcium carbonate (i.e. component (i)) and the tannin or its derivative (i.e. component (ii)) in specific relative amounts to each other. According to one embodiment, the surface-reacted calcium carbonate and the tannin or the derivative thereof are present in the composition in a weight ratio of from 99.9:0.1 to 5:95, preferably from 99:1 to 15:85, more preferably from 95:5 to 25:75, even more preferably from 95:5 to 35:65, yet even more preferably from 95:5 to 45:55.

According to one embodiment, the surface-reacted calcium carbonate and the tannin or the derivative thereof are present in the composition in a weight ratio of from 95:5 to >50: <50, and optionally from 85:15 to 65:35 (e.g. from 80:20 to 70:30).

According to one embodiment, the composition comprises components (i) and (ii) in a weight amount of at least 50 wt. % (e.g. 50 to 100 wt. %), preferably at least 75 wt. %, and more preferably at least 95 wt. %, based on the total weight of the composition.

It is possible that the composition according to the invention comprises an additional component in addition to mandatory components (i) and (ii). However, it is also possible, and usually preferred, that the composition according to the invention essentially consists or consists of at least one surface-reacted calcium carbonate and at least one tannin or derivative thereof. According to one embodiment, the composition essentially consists of at least one surface-reacted calcium carbonate and at least one tannin or derivative thereof. “Essentially consisting of” can mean in this context that specific further components can be present which do not affect the essential characteristics of the composition, e.g. the function as defined herein below in connection with the inventive use.

According to one embodiment, the composition consists of at least one surface-reacted calcium carbonate and at least one tannin or derivative thereof. As will be understood by a person of skill, the composition according to the invention can always contain unavoidable trace impurities and/or residual moisture.

The composition according to the invention preferably does not contain an aluminum content of more than 1000 ppm, preferably more than 300 ppm, and more preferably of more than 200 ppm.

According to one embodiment, the composition is a solid particle blend of the surface-reacted calcium carbonate and the tannin or derivative thereof. According to one embodiment, the composition is the surface-reacted calcium carbonate which is loaded with the tannin or derivative thereof.

According to one preferred embodiment, the composition is a solid composition comprising, optionally consisting of, the surface-reacted calcium carbonate being loaded with the tannin or its derivative, wherein the surface-reacted calcium carbonate and the tannin or the derivative thereof are present in the composition in a weight ratio of from 85:15 to 45:55, and preferably 85:15 to 65:35.

According to one preferred embodiment, the composition is a solid composition comprising, optionally consisting of, the surface-reacted calcium carbonate being loaded with the tannin or its derivative, wherein the surface-reacted calcium carbonate and the tannin or the derivative thereof are present in the composition in a weight ratio of from 85:15 to 45:55, and preferably 85:15 to 65:35,
wherein the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment,
and wherein the tannin is selected from the group of gallotannins, mixtures thereof, and derivatives thereof, and preferably is tannic acid.

According to one preferred embodiment, the composition is a solid particle blend comprising, optionally consisting of, the surface-reacted calcium carbonate and the tannin or its derivative, wherein the surface-reacted calcium carbonate and the tannin or the derivative thereof are present in the composition in a weight ratio of from 65:35 to 35:65.

According to one preferred embodiment, the composition is a solid particle blend comprising, optionally consisting of, the surface-reacted calcium carbonate and the tannin or its derivative, wherein the surface-reacted calcium carbonate and the tannin or the derivative thereof are present in the composition in a weight ratio of from 65:35 to 35:65,
wherein the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H₃O⁺ ion donors treatment,
and wherein the tannin is selected from the group of gallotannins, mixtures thereof, and derivatives thereof, and preferably is tannic acid.

According to one embodiment, the composition is suitable for use as a deodorant and/or an antiperspirant. According to one embodiment, the composition is suitable for use as a deodorant. According to one embodiment, the composition is suitable for use as an antiperspirant. According to one embodiment, the composition is suitable for use as a deodorant and an antiperspirant.

### Personal Care Formulation According to the Invention

In one aspect, the present invention provides a personal care formulation comprising the composition according to the invention.

As regards specific embodiments and preferred embodiments of the composition, it is referred to the definitions, specific embodiments, and preferred embodiments of the composition as defined herein above and in the claims, and which are also disclosed in combination with personal care formulation according to the invention.

The personal care formulation may be any personal care formulation for which the composition according to the invention is suitable for use. The personal care formulation is preferably a personal care formulation which is suitable for application onto the skin, e.g. axillary skin, skin of hands, skin of feet, skin of intimate area, etc. Preferably, the personal care formulation is a deodorant and/or antiperspirant formulation.

The components of the personal care formulation, and preferably of the deodorant and/or antiperspirant formulation, can be selected by the person skilled in the art according to the needs.

According to one embodiment, the personal care formulation is free of aluminum compounds and/or zirconium compounds. “Free” of a substance means that the substance is not deliberately added to the personal care formulation, and preferably that the content of the formulation contains less than 0.1 wt. % (i.e. less than 1000 ppm) (e.g. less than 0.05 wt. % (i.e. less than 500 ppm) or less than 0.02 wt. % (i.e. less than 200 ppm) of the substance, based on the total weight of the formulation.

According to one embodiment, the personal care formulation is free of aluminum compounds. According to one embodiment, the personal care formulation is free of zirconium compounds. According to one embodiment, the personal care formulation is free of aluminum compounds and zirconium compounds.

According to one embodiment, the personal care formulation is free of aluminum salts.

According to one embodiment, the personal care formulation is free of aluminum compounds selected from the group consisting of aluminum chloride, alum (e.g. potassium alum), aluminum chlorohydrate, aluminum-zirconium chlorohydrate, and complexes of these compounds with glycine, propylene glycol, and polyethylene glycol.

According to one embodiment, the personal care formulation comprises the composition according to the invention in an amount of between 1 to 30 wt. %, preferably in an amount between 5 to 25 wt. % (e.g. 5 to 15 wt. %).

The personal care formulation may further comprise one or more compounds selected from the group consisting of water, essential oils, fragrances, antibacterial agents, nonionic surfactants (e.g. polyethylene glycol or propylene glycol), ethoxylated alcohols, emollients, humectants, hydrophilic thickening agents, antioxidants, chelating agents, texturizers, preservatives, alcohols, polymers, silicones, emulsifiers, sodium stearate, neutralizing agents and colors.

The personal care formulation, and preferably the deodorant and/or antiperspirant formulation, can be provided in form of a cream, a lotion, an emulsion, a solid stick, and the like. The formulation can be a water-based, oil-based, hydroalcoholic-based or silicone-based formulation.

### Use According to the Invention

One aspect of the present invention provides the use of a composition according to the invention for reducing sweating and/or sweat odor.

As regards specific embodiments and preferred embodiments of the composition, it is referred to the definitions, specific embodiments, and preferred embodiments of the composition as defined herein above and in the claims, and which are also disclosed in combination with the use according to the invention.

One aspect of the present invention provides the use of a composition according to the invention as a deodorant and/or an antiperspirant.

According to one embodiment, the composition is used as part of a personal care formulation according to the invention.

According to one embodiment, the composition is used for reducing sweating and/or sweat odor of a human, optionally a human of an age in the range of 12 to 75 years, optionally of an age in the range of 18 to 75 years, optionally of an age in the range of 25 to 65 years, optionally of an age 35 to 55 years.

According to one embodiment, the composition is used for reducing sweating and/or sweat odor over a period of time of up to 72 hours, optionally up to 48 hours, optionally up to 36 hours, and optionally up to 24 hours.

According to one embodiment, the composition is used for reducing sweating and/or sweat odor, and preferably sweat odor, of a human by at least 10%, preferably at least 15%, and more preferably at least 20%, compared to a human which has not been treated with the composition.

According to one embodiment, the composition is used as part of a personal care formulation as defined herein, and for reducing sweating and/or sweat odor, and preferably sweat odor, of a human by at least 10%, preferably at least 15%, and more preferably at least 20%, compared to a human which has not been treated with the personal care formulation.

According to one embodiment, the composition is used for absorbing body fluids such as sweat.

One aspect of the present invention provides the use of a composition according to the invention for preparing a deodorant and/or antiperspirant formulation.

In the following, the invention is described by examples. The examples are meant to further illustrate the invention but are not to be construed as limiting the invention in any way.

### EXAMPLES

### 1. Measurement Methods

### 1.1 Particle Properties

### Particle Size Distribution

The volume determined median particle size d₅₀(vol) and the volume determined top cut particle size d₉₈(vol) of surface-reacted calcium carbonate (SRCC) was evaluated using a Malvern Mastersizer 3000 Laser Diffraction System (Malvern Instruments Plc., Great Britain). The d₅₀(vol) or d₉₈(vol) value indicates a diameter value such that 50% or 98% by volume, respectively, of the particles have a diameter of less than this value. The raw data obtained by the measurement was analyzed using the Mie theory, with a particle refractive index of 1.57 and an absorption index of 0.005. The methods and instruments are known to the skilled person and are commonly used to determine particle size distributions of fillers and pigments. SRCC was measured in an aqueous solution of 0.1 wt.-% Na₄P₂O₇. The samples were dispersed using a high-speed stirrer. Loaded surface-reacted calcium carbonate (loaded SRCC; e.g. inventive examples IE1 and IE2) was measured in dry condition without any prior treatment at a pressure of 3 bar.

The weight determined median particle size d₅₀(wt) was measured by the sedimentation method, which is an analysis of sedimentation behaviour in a gravimetric field. The measurement was made with a Sedigraph™ 5120 of Micromeritics Instrument Corporation, USA. The method and the instrument are known to the skilled person and are commonly used to determine particle size distributions of fillers and pigments. The measurement was carried out in an aqueous solution of 0.1 wt.-% Na₄P₂O₇. The samples were dispersed using a high-speed stirrer and supersonicated.

### Specific Surface Area

The specific surface area of SRCC was measured via the BET method according to ISO 9277:2010 using nitrogen. The samples were pre-dried in an oven at 200° C. for >4 h. The samples were then degassed in a VacPrep degassing unit for at least 60 min.

The specific surface area of the loaded SRCC (e.g. examples IE1 and IE2) was measured via the BET method according to ISO 9277:2010 using nitrogen. The samples were pre-dried in an oven at 110° C. for >4 h. The samples were then degassed in a VacPrep degassing unit for at least 60 min.

Intra-Particle Intruded Specific Pore Volume (in cm³/g)

The specific pore volume was measured using a mercury intrusion porosimetry measurement using a Micromeritics Autopore V 9620 mercury porosimeter having a maximum applied pressure of mercury 414 MPa (60 000 psi), equivalent to a Laplace throat diameter of 0.004 μm. The equilibration time used at each pressure step was 20 seconds. The sample material was sealed in a 5 cm³ chamber powder penetrometer for analysis. The data were corrected for mercury compression, penetrometer expansion and sample material compression using the software Pore-Comp (Gane, P. A. C., Kettle, J. P., Matthews, G. P. and Ridgway, C. J., “Void Space Structure of Compressible Polymer Spheres and Consolidated Calcium Carbonate Paper-Coating Formulations”, Industrial and Engineering Chemistry Research, 35(5), 1996, p 1753-1764.).

The total pore volume seen in the cumulative intrusion data can be separated into two regions with the intrusion data from 214 μm down to about 1-4 μm showing the coarse packing of the sample between any agglomerate structures contributing strongly. Below these diameters lies the fine inter-particle packing of the particles themselves. If they also have intra-particle pores, then this region appears bi-modal, and by taking the specific pore volume intruded by mercury into pores finer than the modal turning point, i.e. finer than the bi-modal point of inflection, the specific intra-particle pore volume is defined. The sum of these three regions gives the total overall pore volume of the powder, but depends strongly on the original sample compaction/settling of the powder at the coarse pore end of the distribution.

By taking the first derivative of the cumulative intrusion curve the pore size distributions based on equivalent Laplace diameter, inevitably including pore-shielding, are revealed. The differential curves clearly show the coarse agglomerate pore structure region, the inter-particle pore region and the intra-particle pore region, if present. Knowing the intra-particle pore diameter range it is possible to subtract the remainder inter-particle and inter-agglomerate pore volume from the total pore volume to deliver the desired pore volume of the internal pores alone in terms of the pore volume per unit mass (specific pore volume). The same principle of subtraction, of course, applies for isolating any of the other pore size regions of interest.

### Scanning Electron Microscopy (SEM)

A double faced adhesive tape (C-Tape, electrically conducting) was mounted on a sample holder. The powder sample was put on the tape and spread by tapping the sample holder while keeping the sample holder horizontal. A surplus of powder was removed by tapping the holder while holding it at an angle and carefully applying compressed CO2. The stub was then sputtered with 8 nm Au. The investigation under the FESEM (Zeiss Sigma VP) was done at 5 kV using secondary electron detector (SE2).

### 1.2 In Vitro Measurement of Antiperspirant Effect

In vitro measurement of antiperspirant activity was measured with an SOD4 device from Microfactory, France. The SOD4 device is an automated biomimetic system that imitates the sweating process. The device comprises (1) Smart-Pore™: a silicone microfluidic chip containing microchannels which mimic skin pores (number of pores per chip: 4; dimensions of the pores: 60 μm×60 μm×2 mm; material: polydimethylsiloxane); (2) a pressure system controlling the flow of sweat in the pores down to nanoliter volumes; (3) a climate chamber regulated in humidity/temperature to reproduce perspiration conditions; (4) an optical system to monitor and record the interaction between the antiperspirant and artificial sweat.

Known antiperspirants block sweat pores thereby reducing sweating of a subject. The blocking is caused by a precipitate or clot in the sweat pore which is formed by interaction of the antiperspirant with components of sweat. The SOD4 device mimics this process and allows for analyzing the interaction of an antiperspirant sample and artificial sweat in the microfluidic channel. The analysis includes, e.g. detection of precipitate/clot formation, measuring precipitate/clot formation time, and measuring pressure (bursting pressure) which is necessary for removing the precipitate/clot from the microchannel.

Table 1 shows the specification of the SOD4 device used for the experiments.

TABLE 1

| | |
|---|---|
| Climatic specification | |
| Temperature range | from ambient to 38° C. |
| Humidity range | from ambient to 90% |
| Fluidic specification | |
| Pressure supply requested | 5 bar |
| Pressure range | from 0 to 4 bar |
| Flow-rate range | From 0 to 7 microliters/minutes |
| Optical specifications | |
| Resolution | 5 megapixels |
| Magnification | ×200 |
| Video frequency | One image each (between 1 minute and 30 |
| | seconds) during waiting time; one image |
| | each 25 ms during unclogging time |

Table 2 shows the composition of the artificial sweat used in the in vitro experiments.

TABLE 2

| Component | Quantity (w/v) |
|---|---|
| Sodium chloride | 0.5% |
| Lactic acid | 0.1% |
| Urea | 0.1% |
| Bovine serum albumin | 0.1% |
| Millipore water | up to 100% |
| Fluorescein | 0.1% |
| Ammonium hydroxide | adjusted to |
| | pH = 6.5 |

The following general test protocol was used:
(1) Preparation of a batch of artificial sweat for each new sample;
(2) Injection of the sweat into Smart-Pore™ microchannels of SOD4 instrument and stabilization of the interface on the surface;
(3) Deposition of antiperspirant on the Smart-Pore™ microchannel outlets (2 mg/cm² dose);
(4) Antiperspirant/sweat interaction for one hour at 25° C. temperature and 80% hygrometry (optical monitoring);
(5) Pressure ejection of the formed precipitate/clot after one hour of contacting time.
(6) Analysis and report of the results.

### 1.3 In Vivo Measurement of Deodorant Effect

The deodorant and/or antiperspirant effect a personal care formulation was tested with human volunteers. All volunteers were informed about the scope and importance of the study and gave their written informed consent.

Volunteers were chosen according to the following inclusion criteria:
at least 18 years (female and male)
clinically healthy
well perceivable axillary sweat odor in both axilla

Rejection criteria for volunteers were:
skin diseases or skin allergies
any dermatological, physical or medic conditions possibly interfering with the study
not sufficiently perceivable axillary sweat odor
smokers

Volunteers could stop participating in the study at any time without giving a reason. The intensity of sweat odor and fragrance/inherent smell was assessed olfactorily by an expert panel. A test item was tested in comparison to the untreated contralateral axilla or a reference item. In the initial preconditioning phase over several days the volunteers only used a perfume-free, skin-pH-neutral liquid soap free of bactericidal ingredients and no deodorants or antiperspirants. This soap was also used during the test phase. Before and after randomized test item application (deodorants/antiperspirants) sweat odor intensity and fragrance intensity were determined olfactorily (in-direct sniffing) by three trained experts. For the assessment of the duration of the efficacy different time points were chosen.

The intensity of sweat odor or fragrance was rated according to the following scale:
1=not detectable
2=low
3=detectable
4=strong
5=extremely strong

### 2. Materials

### 2.1 Raw Materials

### Surface-Reacted Calcium Carbonate (SRCC):

SRCC had a d₅₀(vol)=7.8 μm, d₉₈(vol)=20 μm, SSA=96 m2/g with an intra-particle intruded specific pore volume of 1.184 cm³/g (for the pore diameter range of 0.004 to 0.43 μm). SRCC provides a ratio of hydroxyapatite to calcium carbonate of about 85:15 as determined by XRD.

SRCC was obtained by preparing 2000 liters of an aqueous suspension of ground calcium carbonate in a mixing vessel by adjusting the solids content of a ground limestone calcium carbonate from Orgon, France having a mass based particle size distribution of 80% less than 1 μm, as determined by sedimentation, such that a solids content of 13 wt. %, based on the total weight of the aqueous suspension, is obtained.

Whilst mixing the slurry, 715 kg of an aqueous phosphoric acid solution was added over 15 minutes, wherein said solution contained 20 wt. % phosphoric acid. The temperature of the suspension was maintained at 70° C. After the addition of the acid, the suspension was stirred for a minimum of 5 minutes before removing it from the vessel and drying. Tannic acid: Reagent Grade Sigma Aldrich (403040).

### Hydroxyapatite (HAP):

HAP purchased from Sigma Aldrich; particle size distribution d₅₀: 4 to 6 μm; specific surface area (BET): >100 m²/g.

### Ground Natural Calcium Carbonate (GCC):

Ground natural calcium carbonate (marble from Turkey); volume median particle size d₅₀: 5.5 μm; specific surface area (BET) 4 m²/g, available from Omya, Switzerland.

### Tannic Acid:

Tannic acid (CAS: 14001-55-4); reagent grade; purchased from Sigma Aldrich.

### 2.2 Tannic Acid-Loaded SRCC

SRCC Loaded with 25 wt. % Tannic Acid (Inventive Example IE1):

37.5 g of tannic acid was dissolved in 450 ml of deionized water. 112.5 g of SRCC was added to the solution and stirred continuously. The concentration of the active and carrier in the slurry was 25 wt. %. The slurry was dried in a spray dryer (Buchi Mini Spray Dryer B-290 with Inert Loop B-295 and Dehumidifier B-296) using nitrogen (N2) as the spray gas. The feed flow was set to approx. 7.5 ml/min and the spray gas flow to approx. 800-1100 l/h. The inlet temperature was set to 220° C. in order to keep the outlet temperature at approximately 110° C. The spray dried sample was collected and immediately stored in a closed container.

SRCC Loaded with 50 wt. % Tannic Acid (Inventive Example IE2):

75 g of tannic acid was dissolved in 450 ml of deionized water. 75 g of SRCC was added to the solution and stirred continuously. The concentration of the active and carrier in the slurry was 25 wt. %. The slurry was dried in a spray dryer (Buchi Mini Spray Dryer B-290 with Inert Loop B-295 and Dehumidifier B-296) using nitrogen (N2) as the spray gas. The feed flow was set to approx. 7.5 ml/min and the spray gas flow to approx. 800-1100 l/h. The inlet temperature was set to 220° C. to keep the outlet temperature at approximately 110° C. The spray dried sample was collected and immediately stored in a closed container.

TABLE 3
Physical properties of inventive examples IE1 and IE2

| | | | | | | Intraparticle | |
|---|---|---|---|---|---|---|---|
| | | Amount pf | | | | volume [cm³/g] | |
| Inventive | | tannic acid | D50 | D98 | SSA | for the range | d* |
| example | Carrier | [wt. %] | [μm] | [μm] | [m²/g] | 0.004-d*μm | [μm] |
| IE1 | SRCC | 25 | 6.0 | 22 | 49 | 0.834 | 0.40 |
| IE2 | SRCC | 50 | 7.6 | 23 | 17.5 | 0.455 | 0.40 |

### 2.3 Preparation of Solid Particle Blends

Solid particle blends were prepared by adding the respective solid powders into a container (500 ml beaker) followed by thoroughly mixing (manually) for about 15 minutes. The following solid particle blends were prepared:
Tannic acid+SRCC (50:50); inventive example IE3
Tannic acid+solid mixture of 85 parts HAP and 15 parts GCC (50:50); comparative example CE3
Tannic acid+GCC (50:50); comparative example CE4
Tannic acid+HAP (50:50); comparative example CE5

### 3. Test Results

### 3.1 In Vitro Measurement of Antiperspirant Effect

The interaction between active agents and artificial sweat was tested by the method as described above.

The samples used for the tests are shown in Table 4. The solid base materials were diluted in water and mixed for 15 minutes before carrying out the test protocol.

TABLE 4

| | | | | | | Weight |
|---|---|---|---|---|---|---|
| | | | | | Use level | amount |
| | | | | | (weight | tannic |
| | | | | | amount of | acid in |
| | Base materials | | | | material | final |
| Sample | | | Type of | Sample | in final | sample |
| No. | Example | Components | composition | preparation | sample) | (wt. %) |
| Sample 1 | IE1 | SRCC + tannic acid | 25 wt. % | 20 g diluted in | 20% | 5 |
| | | | tannic acid | 80 g water | | |
| | | | loaded on SRCC | | | |
| Sample 2 | IE2 | SRCC + tannic acid | 50 wt. % | 10 g diluted in | 10% | 5 |
| | | | tannic acid | 90 g water | | |
| | | | loaded on SRCC | | | |
| Sample 3 | IE3 | SRCC + tannic acid | Solid particle | 10 g diluted in | 10% | 5 |
| | | | blend (50:50) | 90 g water | | |
| Sample 4 | CE1 | Aluminium | — | 10 g diluted in | 10% | 0 |
| | | chlorohydrate | | 90 g water | | |
| Sample 5 | CE2 | SRCC | — | 5 g diluted in | 5% | 0 |
| | | | | 95 g water | | |
| Sample 6 | CE3 | HAP/GCC (85/15 | Solid particle | 10 g diluted in | 10% | 5 |
| | | solid particle | blend (50:50) | 90 g water | | |
| | | blend) + tannic acid | | | | |
| Sample 7 | CE4 | GCC + tannic acid | Solid particle | 10 g diluted in | 10% | 5 |
| | | | blend (50:50) | 90 g water | | |
| Sample 8 | CE5 | HAP + tannic acid | Solid particle | 10 g diluted in | 10% | 5 |
| | | | blend (50:50) | 90 g water | | |

Furthermore, three commercial deodorant/antiperspirant formulations were tested:
Sample 9 (comparative example CE6): Dove™ Spray 24h (containing aluminum potassium sulfate (alum))
Sample 10 (comparative example CE7): Dove™ Roll-On 24 (containing aluminum potassium sulfate (alum))
Sample 11 (comparative example CE8): Dove™ Roll-On original 48h (containing aluminium chlorohydrate)

The results of the in vitro tests are shown in Table 5.

TABLE 5

| | | Burst pressure | Clot formation |
|---|---|---|---|
| Sample No. | Example | (mbar)* | time (s) |
| Sample 1 | IE1 (inventive) | 1580 +/− 218 | 1810 |
| Sample 2 | IE2 (inventive) | 1259 +/− 150 | 2149 |
| Sample 3 | IE3 (inventive) | 1006 +/− 52 | 1842 |
| Sample 4 | CE1 | 699 +/− 34 | 2047 |
| Sample 5 | CE2 | 0 | No clot formed |
| Sample 6 | CE3 | 828 +/− 100 | 2179 |
| Sample 7 | CE4 | 828 +/− 164 | 2712 |
| Sample 8 | CE5 | 947 +/− 74 | 2361 |
| Sample 9 | CE6 | 689 | N/A |
| Sample 10 | CE7 | 463 | N/A |
| Sample 11 | CE8 | 705 | N/A |

*the burst pressure is the average of the maximal pressure applied before unclogging of precipitate/clot from the microchannel.
** each clot has been observed and the formation time has been calculated with the average of all.

The test results show that the samples comprising the inventive compositions IE1 to IE3 have the highest bursting pressure, which means that for these examples the highest pressure had to be applied to remove the antiperspirant/sweat clot from the microchannel. Thus, the clot formed by the inventive compositions IE1 to IE3 has the highest resistance to removal, which indicates a high efficiency of the inventive compositions in the in vitro test. The samples comprising the inventive compositions IE1 to IE3 also showed the lowest clot formation time, which shows a high activity of the composition in a short period of time compared to other samples. Inventive examples IE1 and IE2 which used tannic acid-loaded SRCC performed even better than a solids blend of tannic acid and SRCC (IE3).

3.1 In Vivo Measurement of Deodorant and/or Antiperspirant Effect

Several deodorant/antiperspirant active agents were formulated in a basic deodorant formulation.

Materials which were used as active agents for the deodorant formulations are shown in Table 6 and the complete formulation recipe of the deodorant formulations is shown in Table 7.

TABLE 6

| | | | | Use level |
|---|---|---|---|---|
| | Base materials | | | (weight amount |
| Sample | | | Type of | of material in |
| name | Example | Components | composition | formulation) |
| Sample A | IE2 | SRCC + | 50 wt. % | 10% |
| | | tannic acid | tannic acid | |
| | | | loaded on | |
| | | | SRCC | |
| Sample B | IE3 | SRCC + | Solid particle | 10% |
| | | tannic acid | blend (50:50) | |
| Sample C | CE5 | HAP + | Solid particle | 10% |
| | | tannic acid | blend (50:50) | |

TABLE 7

| INCI Name | Trade Name | Supplier | Wt. % |
|---|---|---|---|
| Caprylic/Capric | Crodamol | Croda | 60.00 |
| Triglycerides | GTCC | | |
| Cocos Nucifera | Coconut Oil 92 | Bulk Apoth | 7.00 |
| (Coconut) Oil | Degrees | | |
| Ozokerite | Ozokerite White | Strahl & Pitsch | 17.00 |
| | Wax SP 1020P | | |
| Sunflower | Kahlwax 6607L | Omya | 1.00 |
| Seed Wax | MB | | |
| Hydrogenated | Kahlwax 6240 | Omya | 5.00 |
| Vegetable Oil | | | |
| Base material | | | 10.00 |
| (according to | | | |
| Table 6) | | | |

Sample formulations based on the recipe shown in Table 7 were prepared according to the process shown in Table 8:

TABLE 8

| Step | Action |
|---|---|
| 1 | Add the caprylic/capric triglycerides to a jacketed mixing vessel |
| | equipped with a turbine style mixer. |
| 2 | Begin heating to 85° +/− 5° C. |
| 3 | Slowly add the tannic acid and the mineral (i.e. the base material). |
| | Mix until homogenous and free of any lumps. |
| 4 | Once the temperature is reached add the coconut oil and |
| | hydrogenated vegetable oil. Mix until melted. |
| 5 | Add the ozokerite to the batch. Mix until totally melted. |
| 6 | Add the sunflower seed wax to the batch. Mix until totally melted. |
| 7 | Begin cooling the batch to 70° C. +/− 5° C. |
| 8 | Mix for 15 minutes and then fill into canisters. |

The test results obtained by the expert panel is shown in Table 9.

TABLE 9

| Sample | Base | Sweat odor | Sweat odor | Sweat odor |
|---|---|---|---|---|
| name | material | (t = 0 h) | (t = 24 h) | reduction (%) |
| Sample A | IE3 | 4.1 | 2.7 | 34.15 |
| Sample B | CE5 | 4.1 | 3.3 | 19.51 |
| Sample A | IE3 | 3 | 2.2 | 26.67 |
| Untreated | — | 3 | 2.9 | 3.33 |
| Sample C | IE2 | 3 | 2.3 | 23.33 |
| Untreated | — | 3 | 2.6 | 13.33 |

The test results show that the formulations comprising the inventive compositions IE2 and IE3 showed a higher reduction of sweat odor than the formulation comprising the comparative composition CE5. A low sweat odor reduction was observed was untreated volunteers.

1. A composition comprising
(i) a surface-reacted calcium carbonate, wherein the surface-reacted calcium carbonate is a reaction product of ground natural calcium carbonate (GNCC) or precipitated calcium carbonate (PCC) with carbon dioxide and one or more H₃O⁺ ion donors and wherein the carbon dioxide is formed in situ by the H₃O⁺ ion donors treatment and/or is supplied from an external source, and
(ii) a tannin or a derivative thereof.

2. The composition according to claim 1, wherein the composition is a solid composition, and/or wherein the composition essentially consists of at least one surface-reacted calcium carbonate and at least one tannin or derivative thereof.

3. The composition according to claim 1, wherein the surface-reacted calcium carbonate is loaded with the tannin or the derivative thereof, optionally by a process comprising the steps of: (a) providing a liquid composition comprising the surface-reacted calcium carbonate and the tannin or the derivative thereof, and (b) drying the liquid composition provided in step (a) to obtain a solid composition.

4. The composition according to claim 1, wherein the surface-reacted calcium carbonate and the tannin or the derivative thereof form a solid particle blend.

5. The composition according to claim 1, wherein the surface-reacted calcium carbonate and the tannin or the derivative thereof are present in the composition in a weight ratio of from 99:1 to 25:75, and preferably from 95:5 to 35:65.

6. The composition according to claim 1, wherein the one or more H3O+ ion donors are selected from the group consisting of hydrochloric acid, sulfuric acid, sulfurous acid, phosphoric acid, citric acid, oxalic acid, an acidic salt, acetic acid, formic acid, and mixtures thereof.

7. The composition according to claim 1, wherein the surface-reacted calcium carbonate is a reaction product of ground natural calcium carbonate (GNCC) with carbon dioxide and one or more H₃O⁺ ion donors, wherein the one or more H₃O⁺ ion donor is phosphoric acid, and wherein the carbon dioxide is formed in situ by the H3O⁺ ion donors treatment.

8. The composition according to claim 1, wherein the surface-reacted calcium carbonate has one or more of the following characteristics, and preferably has the following characteristics:
(i) a volume median particle size (d₅₀) from 1.0 to 75 μm, preferably from 2 to 50 μm, more preferably 3 to 40 μm, even more preferably from 4 to 30 μm, and most preferably from 5 to 15 μm,
(ii) a top cut volume particle size (d₉₈) from 2 to 150 μm, preferably from 4 to 100 μm, more preferably 6 to 80 μm, even more preferably from 8 to 60 μm, and most preferably from 10 to 30 μm,
(iii) a specific surface area (BET) from 15 to 200 m²/g, preferably of from 27 to 180 m2/g, more preferably from 30 to 160 m2/g, even more preferably 45 to 150 m2/g, and most preferably from 48 to 140 m²/g, as measured by the BET method, and
(iv) an intra-particle intruded specific pore volume in the range from 0.10 to 2.3 cm³/g, more preferably from 0.20 to 2.0 cm³/g, even more preferably from 0.40 to 1.8 cm³/g and most preferably from 0.60 to 1.6 cm³/g, calculated from mercury porosimetry measurement.

9. The composition according to claim 1, wherein the surface-reacted calcium carbonate comprises calcium carbonate and hydroxyapatite (HAP), optionally in a weight ratio in the range of 90:10 to 10:90, preferably in the range of 80:20 to 10:90, more preferably in the range of 60:40 to 10:90, even more preferably in the range of 40:60 to 10:90, and most preferably in the range of 25:75 to 10:90.

10. The composition according to claim 1, wherein the tannin is selected from the group consisting of gallotannins, mixtures thereof, and derivatives thereof, or wherein the tannin is selected from the group of hydrolysable tannins, mixtures thereof, and derivatives thereof.

11. The composition according to claim 1, wherein the tannin is tannic acid or a derivative thereof.

12. A personal care formulation comprising the composition according to claim 1.

13. The personal care formulation according to claim 12, wherein the formulation is a deodorant and/or antiperspirant formulation.

14. The personal care formulation according to claim 12, wherein the formulation is free of aluminum compounds and/or a zirconium compounds.

15. A method for reducing sweating and/or sweat odor comprising applying the composition of claim 1 to skin of a subject.
